(19) United States
(12) Patent Application Publication
(10) Pub. No.: US 2024/0350300 A1
(43) Pub. Date: Oct. 24, 2024

(54) Hand warmer with double-sided heating

(71) Applicant: Shenzhen Street Cat Technology Co., Ltd., Shenzhen (CN)

(72) Inventor: Zeda HU, Shenzhen (CN)

(73) Assignee: Shenzhen Street Cat Technology Co., Ltd., Shenzhen (CN)

(21) Appl. No.: 18/596,634

(22) Filed: Mar. 6, 2024

(30) Foreign Application Priority Data
Apr. 19, 2023 (CN) 202320984461.1

(51) Int. Cl. A61F 7/00

(52) U.S. Cl. CPC A61F 7/007; A61F 2007/0036; A61F 2007/0078

(57) ABSTRACT

A hand warmer with double-sided heating is provided. The hand warmer includes a hand warmer body with a cambered surface on one side and a flat surface on the other side. Through the flat surface of the hand warmer body, two hand warmer bodies can be attached together. The hand warmer body includes an outer shell, a heating film disposed on each of two sides of the outer shell, and an aluminum alloy thermally-conductive cover plate covered on the heating film. The outer shell is internally provided with a Hall switch that is tightly attached to the flat surface and a magnetic steel located on a side of the Hall switch. Flat surfaces of the two hand warmer bodies are opposite and attached to each other.

### CROSS-REFERENCE TO THE RELATED APPLICATIONS

This application is based upon and claims priority to Chinese Patent Application No. 202320984461.1, filed on Apr. 19, 2023, the entire contents of which are incorporated herein by reference.

### TECHNICAL FIELD

The present disclosure relates to the technical field of living supplies, and specifically, to a hand warmer with double-sided heating.

### BACKGROUND

A hand warmer is an automatic heating and health care product. This product is highly popular due to its new and unique advantages such as automatic heating, fun, and practicality.

A hand warmer with a double-sided heating function on the market is used with both hands held together and cannot be separated. Two hand warmers need to be purchased for separate use. However, a plurality of hand warmers are inconvenient to carry and cannot be used in combination, resulting in poor portability.

### SUMMARY

The present disclosure provides a hand warmer with double-sided heating to resolve the technical problem in BACKGROUND.

To achieve the above objective, the present disclosure provides a following technical solution: A hand warmer with double-sided heating includes a hand warmer body with a cambered surface on one side and a flat surface on the other side, and through the flat surface of the hand warmer body, two hand warmer bodies are attached together;
the hand warmer body includes an outer shell, a heating film disposed on each of two sides of the outer shell, and an aluminum alloy thermally-conductive cover plate covered on the heating film; and
the outer shell is internally provided with a Hall switch that is tightly attached to the flat surface and a magnetic steel located on a side of the Hall switch, and when flat surfaces of the two hand warmer bodies are opposite and attached to each other, Hall switches and magnetic steels inside the two hand warmer bodies are opposite, and the Hall switches are triggered.

Further, there is at least one Hall switch and at least one magnetic steel inside the outer shell.

Further, the two sides of the outer shell each are provided with a heat insulation groove, and the heating film is disposed inside the heat insulation groove.

Further, the heat insulation groove, the heating film, and the aluminum alloy thermally-conductive cover plate that are located on a cambered surface of the outer shell are all arc-shaped, while the heat insulation groove, the heating film, and the aluminum alloy thermally-conductive cover plate that are located on a flat surface of the outer shell each have a flat surface.

Further, the heat insulation groove is internally provided with a waterproof ring.

Further, the hand warmer body is internally provided with a battery and a circuit control board electrically connected to the battery, the heating film, and the Hall switch, and the circuit control board is provided with a switch and a charging interface.

Further, the outer shell is provided with a waterproof switch button and a waterproof charging rubber plug that are respectively corresponding to a switch and a charging interface.

Further, the two sides of the outer shell each are provided with a non-slip rubber strip. Further, the flat surface inside the outer shell is provided with a magnet.

Further, the outer shell includes an upper shell and a lower shell, a shell sealing ring is provided between the upper shell and the lower shell, and the shell sealing ring is provided with a rubber strip sealing ring attached to a non-slip rubber strip.

Compared with the prior art, the present disclosure provides a hand warmer with double-sided heating, which has following beneficial effects:

Compared with a commonly used hand warmer on the market, two hand warmers with double-sided heating can be used in combination or separately. When the two hand warmers are used in combination, a circuit of a heating film on a bonding surface is disconnected and no heat is generated, which is safer and more energy-efficient. A magnet is disposed, which ensures stability during bonding and makes it more convenient to use the hand warmer, thereby achieving economic benefits and having a broad application prospect.

### BRIEF DESCRIPTION OF THE DRAWINGS

FIG. 1 is a schematic structural diagram of the present disclosure;

FIG. 2 is a lateral view of a structure of the present disclosure;

FIG. 3 is an exploded view of a structure of the present disclosure;

FIG. 4 is a schematic structural diagram of an upper shell of the present disclosure;

FIG. 5 is a schematic structural diagram of a lower shell of the present disclosure; and

FIG. 6 is a schematic diagram illustrating a combination of two hand warmer bodies of the present disclosure.

In the figures: 1. hand warmer body; 11. outer shell; 111. waterproof switch button; 112. waterproof charging rubber plug; 113. roping piece; 114. Hall switch; 115. magnetic steel; 12. Heat insulation groove; 13. heating film; 14. waterproof ring; 15. aluminum alloy thermally-conductive cover plate; 16. battery; 17. circuit control board; 18. shell sealing ring; 181. rubber strip sealing ring; 19. non-slip rubber strip.

### DETAILED DESCRIPTION OF THE EMBODIMENTS

The technical solutions in the embodiments of the present disclosure will be described below clearly and completely with reference to the accompanying drawings in the embodiments of the present disclosure. Apparently, the described embodiments are merely some, rather than all of the embodiments of the present disclosure. All other embodiments derived from the embodiments of the present disclosure by those of ordinary skill in the art without creative efforts shall fall within the protection scope of the present disclosure.

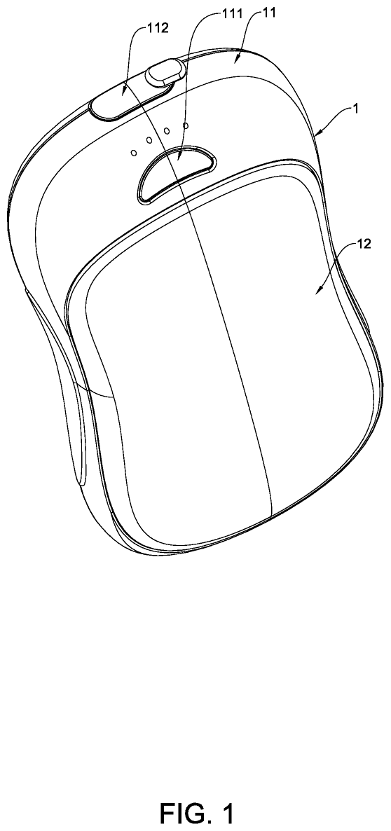

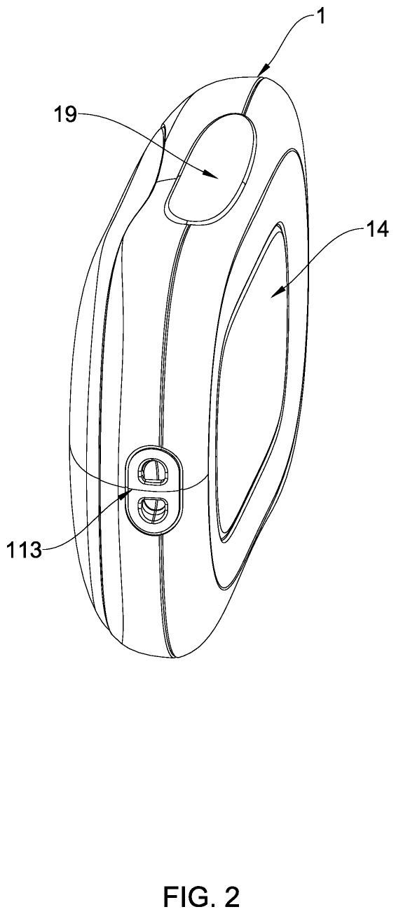

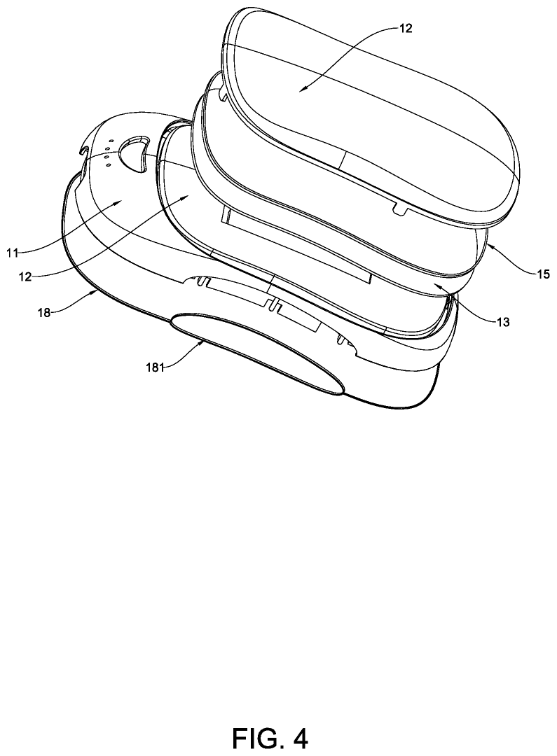

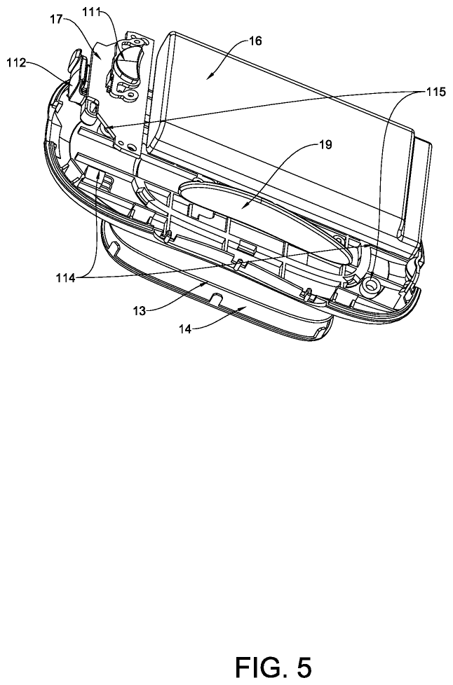

Referring to FIG. 1 to FIG. 6, the present disclosure provides a hand warmer with double-sided heating, including hand warmer body 1. A surface of the hand warmer body 1 is divided into a first surface and a second surface, and through the first surface of the hand warmer body 1, two hand warmer bodies 1 are attached together. The first surface is a flat surface, and the second surface is a curved surface or a cambered surface. The first surface and the second surface are connected to form the surface of the hand warmer body 1.

The hand warmer body 1 includes outer shell 11, heating film 13 disposed on each of two sides of the outer shell 11, and aluminum alloy thermally-conductive cover plate 15 covered on the heating film 13.

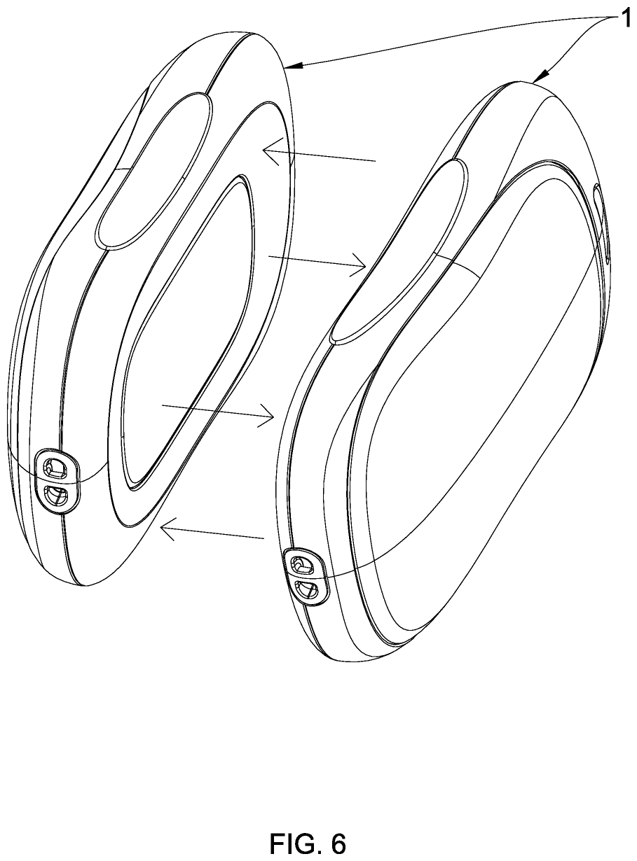

The outer shell 11 is internally provided with Hall switch 114 that is tightly attached to the first surface and magnetic steel 115 located on a side of the Hall switch 114. When the first surfaces of the two hand warmer bodies 1 are opposite and attached to each other, Hall switches 114 and magnetic steels 115 inside the two hand warmer bodies 1 are opposite, and the Hall switches 114 are triggered. A switch-type Hall sensor is mainly configured to measure a quantity of revolutions, a revolving speed, a wind speed, a flow rate, a proximity switch, a door closing notification device, an alarm apparatus, and an automatic control circuit. In this embodiment, the Hall switch 114 is configured to disconnect the heating film 13 located on the flat surface of the outer shell 11, as shown in FIG. 6. When the flat surfaces of the two hand warmer bodies 1 are attached, the Hall switch 114 in one hand warmer body 1 is close to the magnetic steel 115 inside the other hand warmer body 1, and the Hall switch 114 senses a magnetic field of the magnetic steel 115 and controls a circuit of the heating film 13 on the flat surface of the outer shell 11 to be disconnected through a controller on circuit control board 17. This avoids a high temperature generated by opposite heating by the heating films 13 on the flat surfaces of the two hand warmer bodies 1, which is safer and more energy-efficient. One terminal of the outer shell 11 is also provided with a roping piece 113 for connecting a hanging rope.

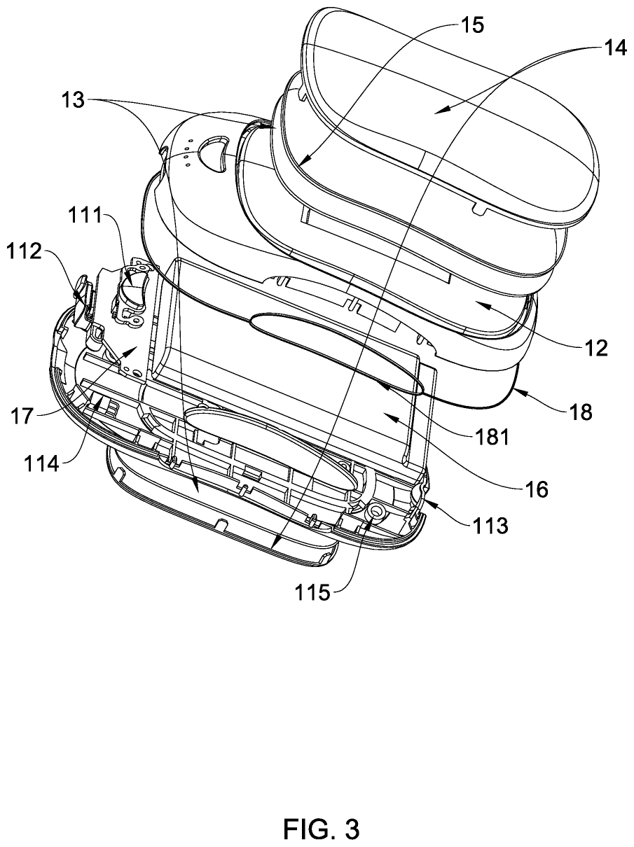

Specifically, there is at least one Hall switch 114 and at least one magnetic steel 115 inside the outer shell 11, as shown in FIG. 3 and FIG. 6. When there are two Hall switches 114 and two magnetic steels 115 inside the outer shell 11, and both the two Hall switches 114 and the two magnetic steels 115 are diagonally arranged, when the flat surfaces of the two hand warmer bodies 1 are oppositely attached, and one of the hand warmer main bodies 1 is rotated by 180 degrees and turned around, the Hall switches 114 and the magnetic steels 115 inside the two hand warmer bodies 1 remain opposite and can still perform sensing to disconnect the circuit.

Specifically, the two sides of the outer shell 11 each are provided with heat insulation groove 12, and the heating film 13 is disposed inside the heat insulation groove 12. With the heat insulation groove 12, the overhead heating film 13 is away from battery 16, reducing an impact of heat on the battery 16.

Specifically, the heat insulation groove 12, the heating film 13, and the aluminum alloy thermally-conductive cover plate 15 that are located on a cambered surface of the outer shell 11 are all arc-shaped, while the heat insulation groove 12, the heating film 13, and the aluminum alloy thermally-conductive cover plate 15 that are located on a flat surface of the outer shell 11 each have a flat surface. The heat insulation groove 12 is internally provided with waterproof ring 14, which is used for sealing treatment between the aluminum alloy thermally-conductive cover plate 15 and the heat insulation groove 12.

Specifically, the hand warmer body 1 is internally provided with the battery 16 and the circuit control board 17 electrically connected to the battery 16, the heating film 13, and the Hall switch 114, and the circuit control board 17 is provided with a switch and a charging interface.

Specifically, the outer shell 11 is provided with waterproof switch button 111 and waterproof charging rubber plug 112 that are respectively corresponding to the switch and the charging interface. The waterproof switch button 111 and the waterproof charging rubber plug 112 are respectively used for waterproof treatment of the switch and the charging interface.

Specifically, the two sides of the outer shell 11 each are provided with non-slip rubber strip 19. The non-slip rubber strip 19 provides more comfortable grasping, absorbs sweat, and is not easy to slip off.

Specifically, a magnet is provided on the flat surface inside the outer shell 11. Through the magnet, the two hand warmer bodies 1 can be tightly adhered together. Magnets of the two hand warmer bodies 1 have opposite magnetic properties and attract each other.

Specifically, the outer shell 11 includes an upper shell and a lower shell, shell sealing ring 18 is provided between the upper shell and the lower shell, and the shell sealing ring 18 is provided with rubber strip sealing ring 181 attached to the non-slip rubber strip 19. The shell sealing ring 18 and the rubber strip sealing ring 181 can be used to complete sealing between the upper shell and the lower shell and sealing of the non-slip rubber strip 19.

In conclusion, compared with a commonly used hand warmer on the market, two hand warmers with double-sided heating can be used in combination or separately. When the two hand warmers are used in combination, the circuit of the heating film 13 on a bonding surface is disconnected and no heat is generated, which is safer and more energy-efficient. The magnet is disposed, which ensures stability during bonding and makes it more convenient to use the hand warmer, thereby achieving economic benefits and having a broad application prospect.

Although the embodiments of the present disclosure have been illustrated and described, those of ordinary skill in the art can understand that various changes, modifications, replacements, and alterations may be made to these embodiments without departing from the principle and spirit of the present disclosure, and the scope of the present disclosure is defined by the claims and equivalents thereof.

What is claimed is:

1. A hand warmer with double-sided heating, comprising a hand warmer body, wherein a surface of the hand warmer body is divided into a first surface and a second surface, and through the first surface of the hand warmer body, two hand warmer bodies are attached together;
the hand warmer body comprises an outer shell, a heating film disposed on each of two sides of the outer shell, and an aluminum alloy thermally-conductive cover plate covered on the heating film; and
the outer shell is internally provided with a Hall switch and a magnetic steel, wherein the Hall switch is tightly attached to the first surface, and the magnetic steel is located on a side of the Hall switch, and when the first surfaces of the two hand warmer bodies are opposite and attached to each other, Hall switches and magnetic steels inside the two hand warmer bodies are opposite, and the Hall switches are triggered.

2. The hand warmer with double-sided heating according to claim 1, wherein there is at least one Hall switch and at least one magnetic steel inside the outer shell.

3. The hand warmer with double-sided heating according to claim 1, wherein the two sides of the outer shell each are provided with a heat insulation groove, and the heating film is disposed inside the heat insulation groove.

4. The hand warmer with double-sided heating according to claim 1, wherein the first surface is the flat surface, and the second surface is a cambered surface.

5. The hand warmer with double-sided heating according to claim 3, wherein the heat insulation groove, the heating film, and the aluminum alloy thermally-conductive cover plate that are located on a cambered surface of the outer shell are arc-shaped, while the heat insulation groove, the heating film, and the aluminum alloy thermally-conductive cover plate that are located on a flat surface of the outer shell each have a flat surface.

6. The hand warmer with double-sided heating according to claim 5, wherein the heat insulation groove is internally provided with a waterproof ring.

7. The hand warmer with double-sided heating according to claim 1, wherein the hand warmer body is internally provided with a battery and a circuit control board, wherein the circuit control board is electrically connected to the battery, the heating film, and the Hall switch, and the circuit control board is provided with a switch and a charging interface.

8. The hand warmer with double-sided heating according to claim 7, wherein the outer shell is provided with a waterproof switch button and a waterproof charging rubber plug, wherein the waterproof switch button and the waterproof charging rubber plug are respectively corresponding to a switch and a charging interface.

9. The hand warmer with double-sided heating according to claim 1, wherein the two sides of the outer shell each are provided with a non-slip rubber strip.

10. The hand warmer with double-sided heating according to claim 4, wherein the flat surface inside the outer shell is provided with a magnet.

11. The hand warmer with double-sided heating according to claim 1, wherein the outer shell comprises an upper shell and a lower shell, a shell sealing ring is provided between the upper shell and the lower shell, and the shell sealing ring is provided with a rubber strip sealing ring, wherein the rubber strip sealing ring is attached to a non-slip rubber strip.
